(19) United States
(12) Patent Application Publication
(10) Pub. No.: US 2026/0335122 A1
(43) Pub. Date: Oct. 29, 2026

(54) Mandibular Advancement Device

(71) Applicant: Good Sleep Co Pte Ltd, Singapore (SG)

(72) Inventor: Alwin Ngai, Ryde (AU)

(21) Appl. No.: 19/186,942

(22) Filed: Apr. 23, 2025

(51) Int. Cl. A61F 5/56

(52) U.S. Cl. CPC A61F 5/566

(57) ABSTRACT

A mandibular advancement device (MAD) for repositioning a mandible of a user forwardly to avoid blockage of an upper airway of the user is provided. The MAD has upper and lower dental trays for respectively fitting into upper and lower dental arches of the user, and left and right connecting mechanisms for joining the upper and lower dental trays. Each connecting mechanism has first and second studs respectively protruding from the upper and lower dental trays, and a serpentine-shaped arm engageable with the first and second studs. By using the serpentine-shaped arm instead of a straight arm, the MAD provides user comfort by allowing freedom of oral movement over an additional distance on top of a static advancement distance made by the MAD for advancing the mandible while avoiding a user-discomfort arrangement of using lengthened studs.

### TECHNICAL FIELD

The present invention relates to a mandibular advancement device (MA D) for repositioning a mandible of a user forwardly to avoid blockage of an upper airway of the user.

### BACKGROUND

Obstructive sleep apnea (OSA) is a common sleep disorder characterized by repeated episodes of partial or complete obstruction of an upper airway during sleep. A MAD is a non-invasive oral appliance for treating mild to moderate OSA. The MAD works by repositioning a mandible or a lower jaw forwardly from a normal position thereof to thereby increase dimensions of the upper airway and reduce a likelihood of airway collapse.

FIG. 1 depicts a typical realization of a MAD 100. The MAD 100 is formed with an upper dental tray 120 and a lower dental tray 110 connected together by a pair of connecting mechanisms 130, 140. The upper and lower dental trays 120, 110 are used to cover a user's teeth. Take the (left) connecting mechanism 130 as a representative for illustration. In the connecting mechanism 130, a first stud 131 and a second stud 132 are fixed on the upper dental tray 120 and the lower dental tray 110, respectively. As a practical example for illustration, the first stud 131 is attached to the canine or first premolar area of the upper dental tray 120 while the second stud 132 is attached to the molar area of the lower dental tray 110. An arm 135 engages the first and second studs 131, 132 for connecting the upper and lower dental trays 120, 110. The length of the arm 135 determines an extent of forward advancement of the lower jaw from an upper jaw of the user. The arm 135 is held in tension when the user wears the MAD 100. Thereby, the upper jaw tends to draw the lower jaw forwardly from its normal position. A shorter arm results in more advancement of the lower jaw from its normal position.

When the MAD 100 is used to treat the OSA of the user, a clinician may decide to alter a relative position of the upper and lower dental trays 120, 110 for achieving a desired or optimized treatment effect. In practice, a set of candidate arms of different lengths is usually provided and the arm 240 is selected from the set of candidate arms by a titration process. In the titration process, the suitability of the longest candidate arm is first tested, and then shorter and shorter candidate arms are successively tested until a suitable arm is found.

A part from providing a therapeutic distance of advancing the lower jaw from the upper jaw for treating the OSA, the MAD 100 is required to provide sufficient user comfort when the user wears the MAD 100. Of practical importance, the MAD 100 is required to support an additional forward advancement of, say, 1 cm or less for allowing the user to enjoy the comfort of easily opening his or her mouth. However, the Inventor has found that the provision of additional advancement may not be achievable for respective candidate arms in the set. It is because locations and orientations of the first and second studs 131, 132 are fixed on the upper and lower dental trays 120, 110, and specific locations and orientations optimized for all the respective candidate arms are almost impossible to be identified.

There is a need in the art for an improved MAD that circumvents the above-mentioned problem.

### SUMMARY

A first aspect of the present disclosure is to provide a first MAD for repositioning a mandible of a user forwardly to avoid blockage of an upper airway of the user. An orthogonality condition is utilized in developing the first MAD.

The first MAD comprises an upper dental tray, a lower dental tray, a left connecting mechanism and a right connecting mechanism. The upper and lower dental trays are used for fitting into upper and lower dental arches of the user, respectively. The left and right connecting mechanisms are collectively configured to join the upper and lower dental trays such that the mandible is forwardly repositioned from a normal position in occlusion by an advancement distance when the user wears the first MAD.

An individual connecting mechanism, selected from the left and right connecting mechanisms, comprises a first stud protruding from the upper dental tray, a second stud protruding from the lower dental tray, and an arm engageable with the first and second studs. The arm comprises a first lug for receiving the first stud, and a second lug for receiving the second stud.

Regarding an individual stud receivable by a corresponding lug where the individual stud is selected from the first and second studs, the individual stud includes a waist section over which the corresponding lug is repositionable such that the advancement distance is extendable from a static advancement distance achieved under a static condition of the first MAD by an additional distance. The waist section has a waist-section length. The additional distance is utilizable by the user to allow freedom of oral movement over the additional distance to thereby contribute to user comfort.

Advantageously, the arm in the individual connecting mechanism is serpentine-shaped to orient the first and second lugs such that orthogonality between the waist section and the corresponding lug for the individual stud is achievable under the static condition. It causes respective waist-section lengths required to achieve a given additional distance to be reduced in comparison to corresponding waist-section lengths calculated for a straight arm such that user comfort is earned from utilizing the additional distance while avoiding a user-discomfort arrangement of elongating the waist section for generating the additional distance.

In the first MAD, the first lug defines a first plane on which the first lug is located, and the second lug defines a second plane on which the second lug is located. In certain embodiments, the first and second lugs are oriented such that the first and second planes are parallel and offset from each other with a non-zero separation.

In certain embodiments, the first lug comprises a first opening such that the first stud is inserted into the first opening when the first lug receives the first stud. Similarly, the second lug comprises a second opening such that the second stud is inserted into the second opening when the second lug receives the second stud.

In certain embodiments, each of the first and second openings is an elongated opening for allowing the first and second studs to be respectively slidable inside the first and second openings and thereby causing the additional distance to be manually adjustable when the user wears the first MAD.

In certain embodiments, the corresponding lug is slidable over the waist section of the individual stud such that a lateral movement of the lower dental tray with respect to the upper dental tray is producible. The lateral movement voluntarily generated by the user is utilizable to allow additional freedom of oral movement to thereby contribute to further comfort to the user.

In certain embodiments, the first stud is positioned adjacent to a canine region or a first premolar region of the upper dental tray, and the second stud is positioned adjacent to a molar region of the lower dental tray.

In the individual connecting mechanism, generally, the arm is releasably engageable with each of the first and second studs.

In certain embodiments, the arm in the individual connecting mechanism is rigid.

In certain embodiments, the upper and lower dental trays are made of thermoplastic polymer.

In certain embodiments, the arm is made of one or more of the following materials: polyamide (PA), polyurethane (PU), thermoplastic polyurethane (TPU), polyethylene (PE), methyl methacrylate (PMMA), polypropylene (PP), polycarbonate (PC), polyoxymethylene (POM), polyether ether ketone (PEEK) and silicone.

A second aspect of the present disclosure is to provide a second MAD for repositioning a mandible of a user forwardly to avoid blockage of an upper airway of the user. A non-orthogonality condition is utilized in developing the second MAD.

The second MAD comprises an upper dental tray, a lower dental tray, a left connecting mechanism and a right connecting mechanism. The upper and lower dental trays are used for fitting into upper and lower dental arches of the user, respectively. The left and right connecting mechanisms are collectively configured to join the upper and lower dental trays such that the mandible is forwardly repositioned from a normal position in occlusion by an advancement distance when the user wears the second MAD.

An individual connecting mechanism, selected from the left and right connecting mechanisms, comprises a first stud protruding from the upper dental tray, a second stud protruding from the lower dental tray, and an arm engageable with the first and second studs. The arm comprises a first lug for receiving the first stud, and a second lug for receiving the second stud. The first lug defines a first plane on which the first lug is located. The second lug defines a second plane on which the second lug is located.

Regarding an individual stud receivable by a corresponding lug where the individual stud is selected from the first and second studs, the individual stud includes a waist section over which the corresponding lug is repositionable such that the advancement distance is extendable from a static advancement distance achieved under a static condition of the second MAD by an additional distance. The waist section has a waist-section length. In addition, the waist section of the individual stud makes an inclination angle between an axial direction of the waist section and a normal direction of a corresponding plane defined by the corresponding lug. The additional distance is utilizable by the user to allow freedom of oral movement over the additional distance to thereby contribute to user comfort.

Advantageously, the arm in the individual connecting mechanism is serpentine-shaped for reducing the inclination angle of the individual stud under the static condition to thereby reduce respective waist-section lengths required to achieve a given additional distance in comparison to a reference case of using a straight arm in the individual connecting mechanism. As a result, user comfort is earned from utilizing the additional distance while avoiding a user-discomfort arrangement of elongating the waist section for generating the additional distance.

In the second MAD, the first lug defines a first plane on which the first lug is located, and the second lug defines a second plane on which the second lug is located. In certain embodiments, the first and second lugs are oriented such that the first and second planes are parallel and offset from each other with a non-zero separation.

In certain embodiments, the first lug comprises a first opening such that the first stud is inserted into the first opening when the first lug receives the first stud. Similarly, the second lug comprises a second opening such that the second stud is inserted into the second opening when the second lug receives the second stud.

In certain embodiments, each of the first and second openings is an elongated opening for allowing the first and second studs to be respectively slidable inside the first and second openings and thereby causing the additional distance to be manually adjustable when the user wears the second MAD.

In certain embodiments, the corresponding lug is slidable over the waist section of the individual stud such that a lateral movement of the lower dental tray with respect to the upper dental tray is producible. The lateral movement voluntarily generated by the user is utilizable to allow additional freedom of oral movement to thereby contribute to further comfort to the user.

In certain embodiments, the first stud is positioned adjacent to a canine region or a first premolar region of the upper dental tray, and the second stud is positioned adjacent to a molar region of the lower dental tray.

In the individual connecting mechanism, generally, the arm is releasably engageable with each of the first and second studs.

In certain embodiments, the arm in the individual connecting mechanism is rigid.

In certain embodiments, the upper and lower dental trays are made of thermoplastic polymer.

In certain embodiments, the arm is made of one or more of the following materials: PA, PU, TPU, PE, PMMA, PP, PC, POM, PEEK and silicone.

Other aspects of the present disclosure are disclosed as illustrated by the embodiments hereinafter.

### BRIEF DESCRIPTION OF THE DRAWINGS

FIG. 1 depicts a typical realization of a MAD.

FIG. 2 depicts a diagram for showing a model of a stud as used in the MAD, and for illustrating interaction between the stud and its corresponding lug.

FIG. 3 depicts a first scenario of extending the advancement distance when the longest candidate arm in a set of candidate arms is used as an arm in an individual connecting mechanism of the MAD, where the arm is a conventional, straight arm.

FIG. 4 depicts a second scenario of extending the advancement distance when a candidate arm used as the arm is short enough such that first and second lugs of the arm are highly tilted to occupy entire respective waist sections of the first and second studs, respectively.

FIG. 5 exemplarily depicts a serpentine-shaped arm used in the MAD in accordance with the present disclosure.

FIG. 6 depicts a third scenario same as the second one shown in FIG. 4 except that the candidate arm is the serpentine-shaped arm.

Skilled artisans will appreciate that elements in the figures are illustrated for simplicity and clarity and have not necessarily been depicted to scale.

### DETAILED DESCRIPTION

Throughout the description and the appended claims, the words “comprise” “comprising”, and the like are to be construed in an inclusive sense as opposed to an exclusive or exhaustive sense; that is to say, in the sense of “comprising, but not limited to”.

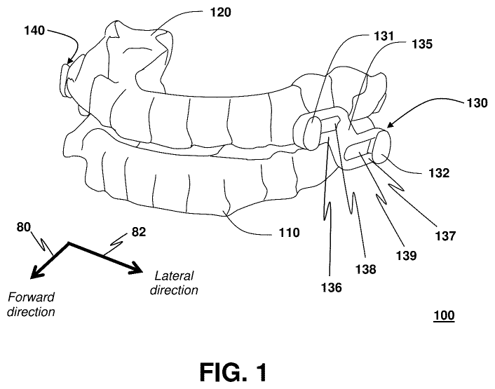

The terms “forward”, “forwarding”, “advance”, “advancement” and the like as used herein in a context refer to the general direction in which a user's face is towards. Consistent with the aforementioned explanation, “a forward direction” is defined herein as a direction 80 shown in FIG. 1 The term “backward” denotes a direction opposite to “forward”. In addition, “a lateral direction” is defined herein as a direction 82 that is a horizontal direction perpendicular to the forward direction 80 as shown in FIG. 1

The term “occlusion” as used herein is interpreted under the technical field of dentistry, and means the way in which upper and lower teeth of a certain person meet.

As used herein, “advancement distance”, or “actual advancement distance”, means a distance that a lower dental tray of a MAD is advanced from a normal position of the lower dental tray, where the normal position of the lower dental tray is determined under a condition that the lower dental tray is aligned with an upper dental tray of the MAD. The lower and upper dental trays are intended to fit into lower and upper dental arches, respectively, of a certain person. That the lower and upper dental trays are aligned corresponds to the situation that the lower and upper dental arches are in normal position in occlusion. Note that the advancement distance is measured along the forward direction 80.

As used herein, “static advancement distance” means an advancement distance produced by a MAD when a pair of connecting mechanisms is deployed in the MAD under a condition or an assumption that an external force for deforming or reshaping the MAD is not exerted to the MAD. In one example, if the MAD is isolated without being worn by a user, the user does not apply a force to a lower or upper dental tray of the MAD so that the advancement distance provided by the MAD is a static advancement distance.

As used herein, “static condition” of a MAD means a condition that the MAD is absent from being exerted with any external force used for deforming or reshaping the MAD.

Before embodiments of the present disclosure are elaborated, an analysis of a conventional design of MAD falling short of providing user comfort is first given as follows.

Refer to FIG. 1. Consider the MAD 100 equipped with the set of candidate arms of different lengths such that the arm 135 is selected from the set of candidate arms by a titration process. As mentioned above, a shorter arm results in a greater advancement of the lower jaw from the upper jaw. Each of the candidate arms, when deployed in the pair of connecting mechanisms 130, 140, causes the MAD 100 to generate a static advancement distance. It is desired to further extend the advancement distance from the static one by an additional distance.

Note that the arm 135 includes a first lug 136 and a second lug 137 respectively located at the two ends of the arm 135. The first lug 136 has a first opening 138 for receiving the first stud 131 Similarly, the second lug 137 has a second opening 139 for receiving the second stud 132 To allow further extending the advancement distance, each of the first and second openings 138, 139 is elongated so that the first and second studs 131, 132 are slidable inside the first and second openings 138, 139, respectively.

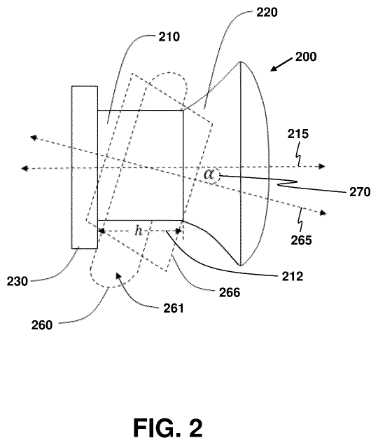

To facilitate explanation of generating the additional distance on top of the static advancement distance, a model of a stud 200 for modeling the first and second studs 131, 132 is first illustrated. The stud 200 is shown in FIG. 2 The stud 200 comprises a first end 220, a second end 230, and a waist section 210 sandwiched between the first and second ends 220, 230. The second end 230 is an interface between the stud 200 and a corresponding dental tray to which the stud 200 is attached. The waist section 210 is narrower than the first and second ends 220, 230, and is used for accommodating a corresponding lug 260 that is inserted to and received by the stud 200. The first end 220 is used for holding the corresponding lug 260 to stay within the waist section 210 when a corresponding arm having the corresponding lug 260 engages the stud 200. The corresponding lug 260 is movable or repositionable over the waist section 210. The waist section 210 has a waist-section length 212 denoted as h and measured along an axial direction 215 of the waist section 210. As will be shown later, the waist-section length 212 is one key parameter in determining the additional distance generated on top of the static advancement distance in the MAD 100.

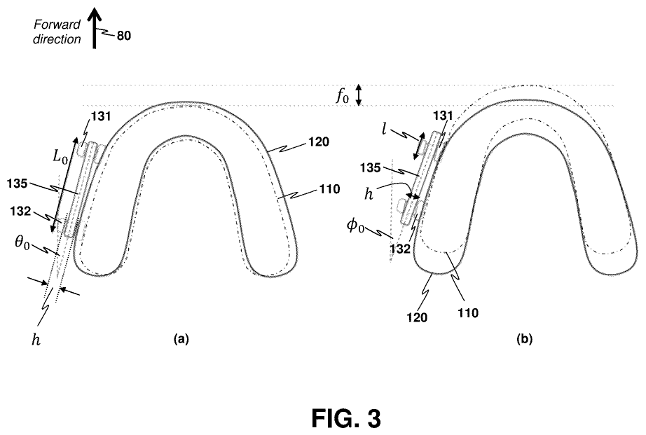

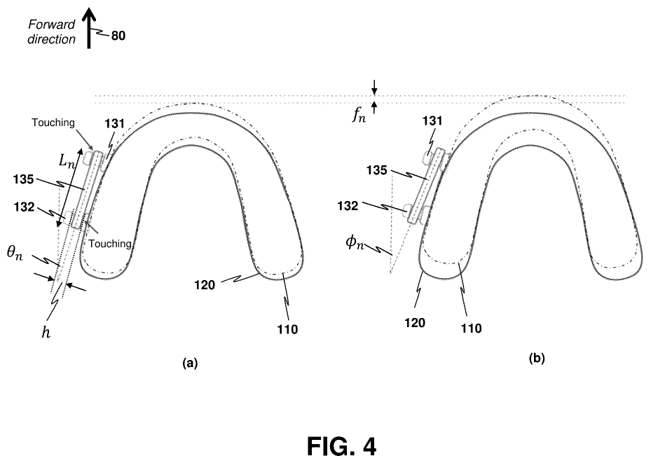

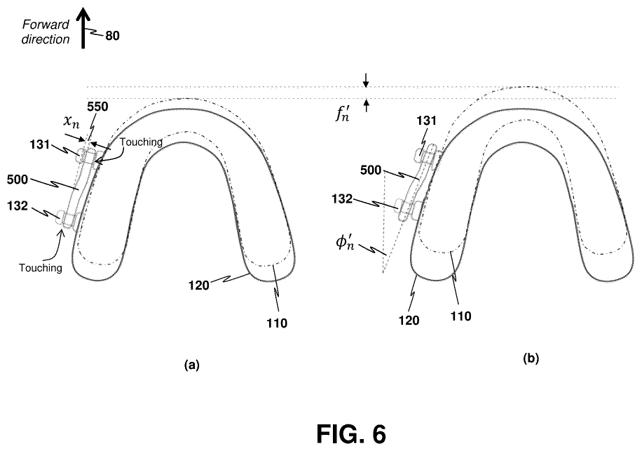

FIG. 3 depicts a first scenario of extending the advancement distance when the longest candidate arm in the set is used as the arm 135. Denote L₀ as the length of the longest candidate arm. Subplot (a) depicts a top view of the MAD 100 under the static condition such that the lower and upper dental trays 110, 120 are aligned and the lower dental tray 110 is located at its normal position. Subplot (b) depicts a top view that the lower dental tray 110 slides forwardly from its normal position to thereby produce an additional distance f₀ on top of the static advancement distance. For simplicity, only the left connecting mechanism 130 is shown in FIG. 3 (and also in FIGS. 4 and 6). It is understood that in practical operations, the left and right connecting mechanisms 130, 140 are present and work together in the MAD 100.

It is first noticed that the arm 135 is not oriented in parallel to the forward direction 80 since the lower and upper dental trays 110, 120, which are intended to respectively fit into an upper dental arch and a lower dental arch of a user of the MAD 100, are curved. In the static condition shown in subplot (a), the arm 135 makes an angle θ₀ with the forward direction 80. Consider the first opening 138, which accommodates the first stud 131, in the following analysis. Denote l as an elongated length of the first opening 138. The first opening 138 as elongated enables realization of the additional distance f₀ for allowing forward/downward movement of the lower jaw of the user in opening the user's mouth. During the movement, the arm 135 starts to tile and finally makes an angle φ₀ with the forward direction 80 where φ₀>θ₀. The waist section 210 of the first stud 131 is required to be sufficiently long (i.e. with a sufficiently large h) to allow this tilting to occur. Since l can be easily made quite large, at a certain point h takes over and becomes a limiting factor in setting an upper bound for φ₀. It is evident that a larger value of h allows a larger difference between φ₀ and θ₀, and hence a larger value of f₀.

In the titration process, the longest candidate arm is first tested, and then shorter and shorter candidate arms in the set are successively tested until a suitable arm for providing an adequate advancement distance required by the user is found. It is possible that before the suitable arm is found, the angle θₙ obtained in testing the nth candidate arm already reaches the above-mentioned upper bound. At this point, further titration is no longer possible.

For illustration, FIG. 4 depicts a second scenario of extending the advancement distance when the nth candidate arm used as the arm 135 is short enough such that the first and second lugs 136, 137 of the arm 135 are highly tilted to occupy entire respective waist sections of the first and second studs 131, 132 respectively. Subplot (a) depicts a top view of the MAD 100 under the static condition while subplot (b) depicts a corresponding top view that the lower dental tray 110 slides forwardly from its normal position to thereby produce an additional distance fₙ on top of the static advancement distance. The arm 135 realized with the nth candidate arm has a length Lₙ that is shorter than L₀. As shown in subplot (a), the first and second lugs 136, 137 touch respective waist-section boundaries of the first and second studs 131, 132 respectively. When the lower jaw slides forwardly as shown in subplot (b), the resultant angle φₙ between the arm 135 and the forward direction 80 is close to θₙ, indicating that the additional distance fₙ is very short. Such short forward movement, fₙ, is obtained since the arm 135 and the first and second lugs 136, 137 interfere each other. Furthermore, such short forward movement is usually obtained only if the user moves his or her upper and lower jaws with great force, causing discomfort to the user.

Based on the forgoing analysis, it is evident that a larger value of h increases the number n, which is indicative to: (1) the maximum static advancement distance achievable by the MAD 100 before the user can comfortably make a small forward movement of his or her lower jaw for comfort or oral relaxation; and (2) the practical number of candidate arms useful for selection of the arm 135. However, increasing h increases a length of each of the first and second studs 131, 132. It has a significant drawback of substantially increasing discomfort to the user. It is highly advantageous if the additional distance can be increased without lengthening the first and second studs 131, 132 in each of the left and right connecting mechanisms 130, 140.

To achieve this advantageous result, the Inventor makes the following observations. For an ith candidate arm, i∈{0, 1 . . . , n}, used as the arm 135, additional tilting from θᵢ to φᵢ is required to generate the additional distance. If it were possible to set θᵢ=0, then φᵢ that could be realized under a given value of h would be a certain maximum value independent of i. The entire waist-section length 212 would be available for tilting the corresponding lug 260 to give φᵢ. Furthermore, achieving φᵢ with this maximum value of angle, which would be involved in tilting the arm 135 to generate the additional distance, would result in a maximally achievable additional distance. The Inventor observes that setting θᵢ=0 for making the entire waist-section length 212 available for tilting is effectively equivalent to arranging the stud 200 and the corresponding lug 260 to be orthogonal to each other. Note that the stud 200 models both cases of the first and second studs 131, 132 The disclosed MAD is developed as follows.

A first aspect of the present disclosure is to provide a MAD for repositioning a mandible of a user forwardly to avoid blockage of an upper airway of the user. The above-mentioned orthogonality condition is utilized in developing the MAD.

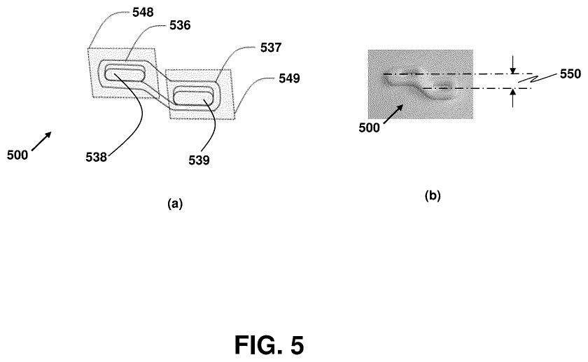

The disclosed MAD has a structure similar to that of the above-mentioned MAD 100 except that an innovative design of the arm 135 as disclosed herein for achieving the above-mentioned orthogonality condition is used in forming the pair of connecting mechanisms 130, 140. The disclosed MAD is exemplarily illustrated with the aid of FIGS. 1 and 5. FIG. 5 exemplarily depicts an arm 500 as used in the disclosed MAD. A prototype of the arm 500 is also shown in FIG. 5 for illustrating a three-dimensional structure of the arm 500. FIG. 1 is reused to illustrate the disclosed MAD except the arm 500. Without causing confusion, the disclosed MAD is referenced as a first MAD 100.

The first MAD 100 as disclosed herein comprises an upper dental tray 120, a lower dental tray 110, a left connecting mechanism 130 and a right connecting mechanism 140. The upper dental tray 120 is used for fitting into an upper dental arch of the user. Similarly, the lower dental tray 110 is used for fitting into a lower dental arch of the user. The left and right connecting mechanisms 130, 140 are collectively configured to join the upper and lower dental trays 120, 110 such that the mandible of the user is forwardly repositioned from a normal position in occlusion by an advancement distance when the user wears the first MAD 100.

In the first MAD 100, the left and right connecting mechanisms 130, 140 share the same structure. Without loss of generality, the left connecting mechanism 130 is described as follows. It is understood that features present in the left connecting mechanism 130 are also present in the right connecting mechanism 140. The left connecting mechanism 130 comprises a first stud 131, a second stud 132, and an arm 500 (as depicted in FIG. 5). The first stud 131 protrudes from the upper dental tray 120. The second stud 132 protrudes from the lower dental tray 110. The arm 500 is engageable with the first and second studs 131 132 Generally, the arm 500 is releasably engageable with each of the first and second studs 131, 132 in that the arm 500 is usually selected from a set of candidate arms and replacement of one candidate arm with another one is made possible. The arm 500 comprises a first lug 536 for receiving the first stud 131, and a second lug 537 for receiving the second stud 132

Note that the first lug 536 comprises a first opening 538 such that the first stud 131 is inserted into the first opening 538 when the first lug 536 receives the first stud 131 Similarly, the second lug 537 comprises a second opening 539 such that the second stud 132 is inserted into the second opening 539 when the second lug 537 receives the second stud 132 Also note that the first lug 536 defines a first plane 548 on which the first lug 536 is located, and that the second lug 537 defines a second plane 549 on which the second lug 537 is located. Specifically, the first and second planes 548, 549 are reference planes defined such that the first plane 548 is coplanar with the first opening 538 and the second plane 549 is coplanar with the second opening 539.

Each of the first and second studs 131, 132 is modeled according to the stud 200 as detailed above with reference to FIG. 2 The stud 200 receivable by its corresponding lug 260 includes the waist section 210 over which the corresponding lug 260 is repositionable. By repositioning one or more of four lugs (viz., respective first and second lugs of the two arms in the left and right connecting mechanisms 130, 140), the advancement distance is extendable from a static advancement distance achieved under a static condition of the first MAD 100 by an additional distance. As mentioned above, the additional distance is utilizable by the user to allow freedom of oral movement over the additional distance to thereby contribute to user comfort.

The waist section 210 is distinguishable from adjacent two ends (namely, the first and second ends 220, 230) in that the waist section 210 is narrower than the two ends 220, 230. The waist section 210 has a waist-section length 212 and an axial direction 215. The waist-section length 212 is a length of the waist section 210 measured along the axial direction 215 of the waist section 210. It can be shown that a length of the additional distance is positively correlated to each of the four waist-section lengths identified in the left and right connecting mechanisms 130, 140.

FIG. 2 depicts a diagram illustrating interaction between the stud 200 and its corresponding lug 260. The corresponding lug 260 engages the stud 200. The corresponding lug 260 has an opening 261 and defines a plane 266 as elaborated above. That is, the first lug 536 defines the first plane 548, and the second lug 537 defines the second plane 549. The plane 266 has a normal direction 265 perpendicular to the plane 266. The stud 200 and the corresponding lug 260 makes an inclination angle 270 denoted as a. The inclination angle 270 is an angle between the axial direction 215 of the waist-section length 212 and the normal direction 265 of the plane 266. The orthogonality condition between the stud 200 and the corresponding lug 260 as mentioned above is satisfied if α=0.

The Inventor identifies that if the arm 500 is not realized as a straight arm but is formed with curvature, it is practically easier to satisfy the above-mentioned orthogonality condition while a manufacturing cost of the arm 500 is usually not sharply increased. Advantageously, the arm 500 in each of the left and right connecting mechanisms 130, 140 is serpentine-shaped to orient the first and second lugs 536, 537 such that orthogonality between the waist section 210 and the corresponding lug 260 for each of the first and second studs is achievable under the static condition. That the aforementioned orthogonality condition is “achievable” is understood that non-orthogonality may result during assembling the first MAD 100 but by actively orienting the first and second lugs 536, 537 in a supervised manner (e.g., with human tuning), the orthogonality condition can be achieved. As a result, it causes the respective waist-section lengths required to achieve a given additional distance to be reduced in comparison to corresponding waist-section lengths calculated for a straight arm. Advantageously, user comfort is earned from utilizing the additional distance while avoiding a user-discomfort arrangement of elongating the waist section 210 for generating the additional distance.

There are many different forms of a serpentine shape. Examples of the serpentine shape include an S shape, a zig-zag shape, an z-shape, and a dog leg shape. Note that the arm 500 as depicted in FIG. 5 has an z-shape, or a zig-zag shape.

Certain forms of the serpentine-shaped arm 500 can be easily manufactured without a significant hike in manufacturing cost in comparison to manufacturing a straight arm. One such form as detailed as follows is shown in FIG. 5. In certain embodiments, the first and second lugs 536, 537 of the serpentine-shaped arm 500 are oriented such that the first and second planes 548, 549 are parallel and offset from each other with a non-zero separation 550. In practical situations, even though the first and second planes 548, 549 are designed to be parallel, the serpentine-shaped arm 500 is usually manufactured with a negligible, non-zero dihedral angle between the first and second planes 548, 549 due to manufacturing tolerances. Generally, the dihedral angle is limited to be not greater than 10°. Herein in the specification and appended claims, the first and second planes 548, 549 are determined to be parallel if the dihedral angle between the first and second planes 548, 549 is not greater than 10°.

To illustrate the advantage of using the serpentine-shaped arm 500 over the straight arm 135 in generating the additional distance, FIG. 6 depicts a third scenario same as the second one considered in FIG. 4 except that the nth candidate arm as used in the first MAD 100 is the serpentine-shaped arm 500 with the non-zero separation 550, xₙ. Subplot (a) depicts a top view of the first MAD 100 under the static condition while subplot (b) depicts a corresponding top view that the lower dental tray 110 slides forwardly from its normal position to thereby produce an additional distance

$${f}_{n}^{′}$$

on top of the static advancement distance. Comparing FIG. 6 against FIG. 4, one finds that there is more spacing on the waist section of the first stud 131 for the serpentine-shaped arm 500 to tile than for the straight arm 135 to tile in case of generating the additional distance. In particular,

$${ϕ}_{n}^{′} ,$$

the angle between the serpentine-shaped arm 500 and the forward direction 80 as shown in FIG. 6, is greater than φₙ. As a result,

$${f}_{n}^{′}$$

is greater than fₙ, thus increasing the additional distance by using the serpentine-shaped arm 500.

Other implementation aspect of the disclosed first MAD 100 are elaborated as follows.

As mentioned above, each of the first and second openings 138, 139 is an elongated opening for allowing the first and second studs 131, 132 to be respectively slidable inside the first and second openings 138, 139. It enables the additional distance to be manually adjustable when the user wears the first MAD 100. A part from this option that both of the first and second openings 138, 139 are elongated, another option is that one of the two openings 138, 139 is elongated while the other one is sized to just accommodate its corresponding stud.

Preferably, the corresponding lug 260 is slidable over the waist section 210 of the stud 200 (modeling each one of the four studs in the left and right connecting mechanisms 130, 140) such that a lateral movement of the lower dental tray 110 with respect to the upper dental tray 120 is producible. The lateral movement is a movement along the lateral direction 82 The lateral movement voluntarily generated by the user is utilizable to allow additional freedom of oral movement to thereby contribute to further comfort to the user.

Usually, the first stud 131 is positioned adjacent to a canine region or a first premolar region of the upper dental tray 120, and the second stud 132 is positioned adjacent to a molar region of the lower dental tray 110. Other tooth positions as deemed appropriate by those skilled in the art according to practical situations may also be used for positioning the first and second studs 131, 132

Preferably, the arm 500 is releasably engageable with each of the first and second studs 131, 132 This feature applies to both the left and right connecting mechanisms 130, 140. This feature enables a suitable serpentine-shaped arm to be selected from a set of candidate arms of different lengths in a titration process.

Generally, the arm 500 is rigid. This feature applies to both the left and right connecting mechanisms 130, 140. Nevertheless, the present disclosure is not limited only to using a rigid arm. In certain embodiments, the arm 500 is semi-rigid or slightly bendable.

In forming the first MAD 100, the arm 500 may be made of a first material more rigid that a second material of the upper and lower dental trays 120, 110 to avoid being readily stretched and losing the therapeutic effect of the first MAD 100. The upper and lower dental trays 120, 110 may be made of thermoplastic polymer. The arm 500 may be made of one or more of the following materials: PA, PU, TPU, PE, PMMA, PP, PC, POM, PEEK and silicone.

The orthogonality condition used in developing the first MAD 100 is an optimal condition for waist-section length minimization. In the orthogonality condition, respective inclination angles (see the inclination angle 270) associated with the four studs in the left and right connecting mechanisms 130, 140 are reduced to zero. The Inventor finds that in a suboptimal non-orthogonality condition that one or more of the respective inclination angles are non-zero, the waist-section length reduction in comparison to the case of using a straight arm can still be achieved.

A second aspect of the present disclosure is to provide a MAD for repositioning a mandible of a user forwardly to avoid blockage of an upper airway of the user. The above-mentioned non-orthogonality condition is utilized in developing the MAD. Without causing confusion, the disclosed MAD is referenced as a second MAD 100.

The second MAD 100 also utilizes the serpentine-shaped arm 500, and is the same as the first MAD 100 except that the serpentine-shaped arm 500 is intended to perform the following function. The serpentine-shaped arm 500 is used for reducing corresponding inclination angles respectively associated with the four studs in the left and right connecting mechanisms 130, 140 to thereby reduce respective waist-section lengths required to achieve a given additional distance in comparison to a reference case of using a straight arm in each of the left and right connecting mechanisms 130, 140. As a result, user comfort is earned from utilizing the additional distance while avoiding a user-discomfort arrangement of waist-section elongation for generating the additional distance.

Accordingly, embodiments of the second MAD 100 follow corresponding embodiments of the first MAD 100 except that the serpentine-shaped arm 500 performs the above-mentioned function. Details of manufacturing the second MAD 100, such as the materials used, follow those of manufacturing the first MAD 100.

The present disclosure may be embodied in other specific forms without departing 1 from the spirit or essential characteristics thereof. The present embodiment is therefore to be considered in all respects as illustrative and not restrictive. The scope of the invention is indicated by the appended claims rather than by the foregoing description, and all changes that come within the meaning and range of equivalency of the claims are therefore intended to be embraced therein.

1. A mandibular advancement device (MAD) for repositioning a mandible of a user forwardly to avoid blockage of an upper airway of the user, the MAD comprising:
an upper dental tray for fitting into an upper dental arch of the user;
a lower dental tray for fitting into a lower dental arch of the user;
left and right connecting mechanisms collectively configured to join the upper and lower dental trays such that the mandible is configured to be forwardly repositioned from a normal position in occlusion by an advancement distance when the user wears the MAD, wherein:
an individual connecting mechanism of the left and right connecting mechanisms comprises a first stud protruding from the upper dental tray, a second stud protruding from the lower dental tray, and an arm engageable with the first and second studs, wherein the arm comprises a first lug for receiving the first stud, and a second lug for receiving the second stud;
an individual stud of the first and second studs receivable by a corresponding lug of the first and second lugs includes a waist section over which the corresponding lug is repositionable such that the advancement distance is extendable from a static advancement distance achieved under a static condition of the MAD by an additional distance, the waist section having a waist-section length, the additional distance being utilizable by the user to allow freedom of oral movement over the additional distance to thereby contribute to user comfort;
the arm of each individual connecting mechanism is shaped with a serpentine shape to orient the first and second lugs such that orthogonality between the waist section and the corresponding lug for the individual stud is achievable under the static condition, causing respective waist-section lengths required to achieve a given additional distance to be reduced in comparison to corresponding waist-section lengths calculated for a straight arm such that user comfort is earned from utilizing the additional distance while avoiding a user-discomfort arrangement of elongating the waist section for generating the additional distance, wherein the serpentine shape is selected from a zig-zag shape, an z-shape and a dog leg shape; and
in each individual connecting mechanism, the first lug comprises a first opening such that the first stud is inserted into the first opening when the first lug receives the first stud, and the second lug comprises a second opening such that the second stud is inserted into the second opening when the second lug receives the second stud, each of the first and second openings being an elongated opening, the first and second studs being respectively slidable inside the first and second openings.

2. The MAD according to claim 1, wherein:
in each individual connecting mechanism, the first lug defines a first plane on which the first lug is located, and the second lug defines a second plane on which the second lug is located, the first and second lugs being oriented such that the first and second planes are parallel and offset from each other with a non-zero separation.

3. (canceled)

4. (canceled)

5. The MAD according to claim 1, wherein in each individual connecting mechanism, the corresponding lug is slidable over the waist section of the individual stud such that a lateral movement of the lower dental tray with respect to the upper dental tray is producible, the lateral movement voluntarily generated by the user being utilizable to allow additional freedom of oral movement to thereby contribute to further comfort to the user.

6. The MAD according to claim 1, wherein in each individual connecting mechanism, the first stud is positioned adjacent to a canine region or a first premolar region of the upper dental tray, and the second stud is positioned adjacent to a molar region of the lower dental tray.

7. The MAD according claim 1, wherein in the individual connecting mechanism, the arm is releasably engageable with each of the first and second studs.

8. The MAD according claim 1, wherein in the individual connecting mechanism, the arm is rigid.

9. The MAD according to claim 1, wherein the upper and lower dental trays are made of thermoplastic polymer.

10. The MAD according to claim 1, wherein the arm of each individual connecting mechanism is made of one or more of the following materials: polyamide (PA), polyurethane (PU), thermoplastic polyurethane (TPU), polyethylene (PE), methyl methacrylate (PMMA), polypropylene (PP), polycarbonate (PC), polyoxymethylene (POM), polyether ether ketone (PEEK) and silicone.

11. A mandibular advancement device (MAD) for repositioning a mandible of a user forwardly to avoid blockage of an upper airway of the user, the MAD comprising:
an upper dental tray for fitting into an upper dental arch of the user;
a lower dental tray for fitting into a lower dental arch of the user;
left and right connecting mechanisms collectively configured to join the upper and lower dental trays such that the mandible is configured to be forwardly repositioned from a normal position in occlusion by an advancement distance when the user wears the MAD, wherein:
an individual connecting mechanism of the left and right connecting mechanisms comprises a first stud protruding from the upper dental tray, a second stud protruding from the lower dental tray, and an arm engageable with the first and second studs, wherein the arm comprises a first lug for receiving the first stud, and a second lug for receiving the second stud, and wherein the first lug defines a first plane on which the first lug is located, and the second lug defines a second plane on which the second lug is located;
an individual stud of the first and second studs receivable by a corresponding lug of the first and second lugs includes a waist section over which the corresponding lug is repositionable such that the advancement distance is extendable from a static advancement distance achieved under a static condition of the MAD by an additional distance, the waist section having a waist-section length and making an inclination angle between an axial direction of the waist section and a normal direction of a corresponding plane defined by the corresponding lug, the additional distance being utilizable by the user to allow freedom of oral movement over the additional distance to thereby contribute to user comfort; and
the arm of each individual connecting mechanism is shaped with a serpentine shape for reducing the inclination angle of the individual stud under the static condition to thereby reduce respective waist-section lengths required to achieve a given additional distance in comparison to a reference case of using a straight arm in the individual connecting mechanism such that user comfort is earned from utilizing the additional distance while avoiding a user-discomfort arrangement of elongating the waist section for generating the additional distance, wherein the serpentine shape is selected from a zig-zag shape, an z-shape and a dog leg shape; and
in each individual connecting mechanism, the first lug comprises a first opening such that the first stud is inserted into the first opening when the first lug receives the first stud, and the second lug comprises a second opening such that the second stud is inserted into the second opening when the second lug receives the second stud, each of the first and second openings being an elongated opening, the first and second studs being respectively slidable inside the first and second openings.

12. The MAD according to claim 11, wherein:
in each individual connecting mechanism, the first and second lugs are oriented such that the first and second planes are parallel and offset from each other with a non-zero separation.

13. (canceled)

14. (canceled)

15. The MAD according to claim 13, wherein the corresponding lug is slidable over the waist section of the individual stud such that a lateral movement of the lower dental tray with respect to the upper dental tray is producible, the lateral movement voluntarily generated by the user being utilizable to allow additional freedom of oral movement to thereby contribute to further comfort to the user.

16. The MAD according to claim 11, wherein in each individual connecting mechanism, the first stud is positioned adjacent to a canine region or a first premolar region of the upper dental tray, and the second stud is positioned adjacent to a molar region of the lower dental tray.

17. The MAD according claim 11, wherein in the individual connecting mechanism, the arm is releasably engageable with each of the first and second studs.

18. The MAD according claim 11, wherein in the individual connecting mechanism, the arm is rigid.

19. The MAD according to claim 11, wherein the upper and lower dental trays are made of thermoplastic polymer.

20. The MAD according to claim 11, wherein the arm of each individual connecting mechanism is made of one or more of the following materials: polyamide (PA), polyurethane (PU), thermoplastic polyurethane (TPU), polyethylene (PE), methyl methacrylate (PMMA), polypropylene (PP), polycarbonate (PC), polyoxymethylene (POM), polyether ether ketone (PEEK) and silicone.
